(19) United States
(12) Patent Application Publication
(10) Pub. No.: US 2021/0096353 A1
(43) Pub. Date: Apr. 1, 2021

(54) ILLUMINATING DEVICE AND ENDOSCOPE SYSTEM

(71) Applicant: OLYMPUS CORPORATION, Tokyo (JP)

(72) Inventor: Kazuaki MURAYAMA, Tokyo (JP)

(73) Assignee: OLYMPUS CORPORATION, Tokyo (JP)

(21) Appl. No.: 17/122,015

(22) Filed: Dec. 15, 2020

Related U.S. Application Data
(63) Continuation of application No. PCT/JP2018/023804, filed on Jun. 22, 2018.

(51) Int. Cl. G02B 23/24

(52) U.S. Cl. CPC G02B 23/2461; G02B 26/0833; G02B 23/243

(57) ABSTRACT

An illuminating device includes: a light source; a light guide element; and a deflecting element that has a deflecting surface for deflecting illumination light coming from the light source towards a light-incident end of the light guide element and that makes the illumination light incident on the light-incident end at incident angles according to deflection angles. The deflecting element is configured to change, between a first deflection angle and a second deflection angle, the deflection angle of the illumination light at each position on the deflecting surface and deflects the illumination light simultaneously at the first deflection angle and at the second deflection angle. A first portion deflected at the first deflection angle and a second portion deflected at the second deflection angle are both incident on the light-incident end.

### CROSS-REFERENCE TO RELATED APPLICATIONS

This is a continuation of International Application PCT/JP2018/023804, with an international filing date of Jun. 22, 2018, which is hereby incorporated by reference herein in its entirety.

### TECHNICAL FIELD

The present invention relates to an illuminating device and an endoscope system.

### BACKGROUND ART

There are well-known endoscope illuminating devices that control the light distribution pattern of illumination light by means of a digital mirror array device (DMD®) and that supply the illumination light to an endoscope via a lightguide (for example, refer to PTL 1). If partially intense reflection light occurs on a subject, an image of the subject becomes partially bright due to halation or a bright spot on the image, and this makes it difficult to observe the subject. In observing, for example, the interior of a body cavity, as a result of a cavity wall in the vicinity of the distal end of the endoscope being irradiated with intense illumination light, halation occurs at a peripheral portion of the image. When illumination light that is regularly reflected at a surface of the subject is incident on an objective lens of the endoscope, a bright spot occurs in the image. Such halation and a bright spot can be suppressed by controlling the light distribution pattern of the illumination light by means of a DMD such that a region in which the halation or the bright spot occurs is illuminated with a moderate level of illumination light.

### CITATION LIST

### Patent Literature

### {PTL 1}

Publication of Japanese Patent No. 4588843

### SUMMARY OF INVENTION

One aspect of the present invention is directed to an illuminating device including: a light source; a light guide element that has a light-incident end and a light-emitting end, the light guide element guiding illumination light incident on the light-incident end to emit the illumination light from the light-emitting end; and a deflecting element that has a deflecting surface for deflecting the illumination light coming from the light source towards the light-incident end of the light guide element and that makes the illumination light incident on the light-incident end at an incident angle according to a deflection angle, wherein the deflecting element can change, between a first deflection angle and a second deflection angle, the deflection angle of the illumination light at each position on the deflecting surface and deflects the illumination light simultaneously at the first deflection angle and the second deflection angle, and a first portion of the illumination light, the first portion being deflected at the first deflection angle, and a second portion of the illumination light, the second portion being deflected at the second deflection angle, are both incident on the light-incident end.

Another aspect of the present invention is directed to an endoscope system including: one of the above-described illuminating devices; and an image-capturing unit that captures an image of a subject illuminated with illumination light emitted from the light-emitting end of the light guide element.

### BRIEF DESCRIPTION OF DRAWINGS

FIG. 1 is an overall configuration diagram of an endoscope system according to one embodiment of the present invention.

FIG. 2 is an overall configuration diagram of an illuminating device of the endoscope system in FIG. 1.

FIG. 3A is a diagram illustrating the relationship between the incident angle of illumination light that is incident on a light-incident end of a light guide element and the emission angle of the illumination light that is emitted from a light-emitting end of the light guide element.

FIG. 3B is a diagram depicting one example of illumination light emitted from the light-emitting end of the light guide element.

FIG. 4A is a diagram depicting one example of light distribution characteristics of illumination light that is emitted from the light-emitting end in the case where only on-light from a mirror array device is incident on the light guide element.

FIG. 4B is a diagram depicting one example of light distribution characteristics of illumination light that is emitted from the light-emitting end in the case where only off-light from the mirror array device is incident on the light guide element.

FIG. 4C is a diagram depicting one example of light distribution characteristics of illumination light that is emitted from the light-emitting end in the case where both on-light and off-light from the mirror array device are incident on the light guide element.

FIG. 5 is a front elevational view of the mirror array device.

FIG. 6A is a diagram illustrating an example of controlling the micromirrors of the mirror array device in the case where the ratio between on-light and off-light is controlled to 80:20.

FIG. 6B is a diagram depicting light distribution characteristics of illumination light emitted from the light-emitting end of the light guide element in the control shown in FIG. 6A.

FIG. 7A is a diagram illustrating an example of controlling the micromirrors of the mirror array device in the case where the ratio between on-light and off-light is controlled to 30:70.

FIG. 7B is a diagram depicting light distribution characteristics of illumination light emitted from the light-emitting end of the light guide element in the control shown in FIG. 7A.

FIG. 8A is a diagram illustrating an example of controlling the micromirrors of the mirror array device in the case where the ratio between on-light and off-light is controlled to 15:85.

FIG. 8B is a diagram depicting light distribution characteristics of illumination light emitted from the light-emitting end of the light guide element in the control shown in FIG. 8A.

FIG. 9 is a diagram illustrating another example of controlling the micromirrors of the mirror array device.

FIG. 10 is an overall configuration diagram of a modification of the illuminating device in FIG. 1.

### DESCRIPTION OF EMBODIMENTS

An illuminating device 1 and an endoscope system 100 according to one embodiment of the present invention will now be described with reference to the drawings.

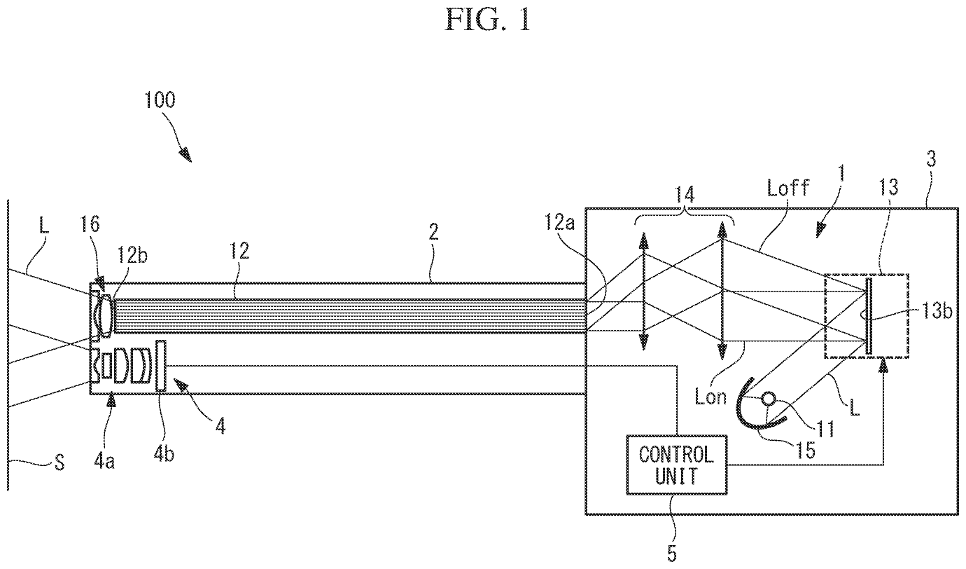

As shown in FIG. 1, the endoscope system 100 according to this embodiment includes an elongated scope 2 and a light source device 3 connected to the basal end of the scope 2. In addition, the endoscope system 100 includes an image-capturing unit 4 for capturing an image of a subject S, the illuminating device 1 for illuminating the field of view of the image-capturing unit 4, and a control unit 5 for controlling the illuminating device 1.

The image-capturing unit 4 includes an image-forming lens 4a and an image sensor 4b. The image-forming lens 4a is disposed on a distal end surface of the scope 2 and images light coming from the subject S. The image sensor 4b is disposed in the scope 2 and captures the image of the subject S formed by the image-forming lens 4a, thereby generating an image. The image of the subject S is transmitted from the image sensor 4b to the control unit 5. The control unit 5 displays the image on a display device (not shown in the figure).

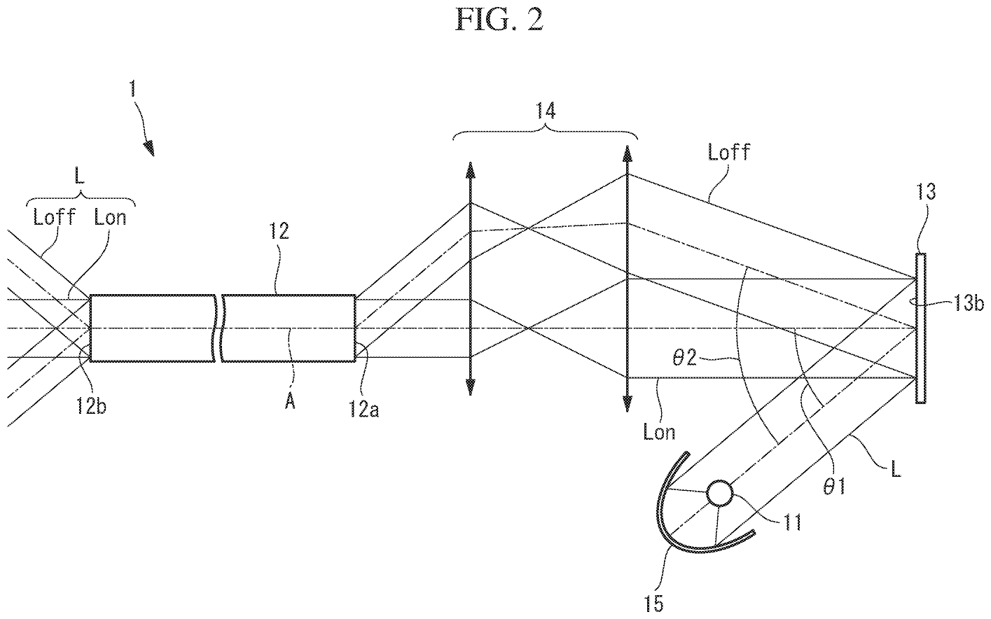

As shown in FIG. 2, the illuminating device 1 includes a light source 11 for emitting illumination light L, a light guide element 12 for guiding the illumination light L, a mirror array device (deflecting element) 13 for deflecting the illumination light L coming from the light source 11 towards a light-incident end 12a of the light guide element 12, and a lens group 14 disposed between the mirror array device 13 and the light guide element 12.

The light source 11, the mirror array device 13, and the lens group 14 are disposed in the light source device 3. The light guide element 12 is disposed in the scope 2 along the longitudinal direction thereof, extending from the basal end of the scope 2 to the vicinity of the distal end of the scope 2.

The light source 11 is a solid-state light source, such as a light-emitting diode (LED). Reference sign 15 is a parabolic surface mirror for collimating the illumination light L, which is a diverging beam emitted from the light source 11, into substantially collimated light. The light source 11 emits the illumination light L in the opposite direction to the mirror array device 13, and the parabolic surface mirror 15 reflects the illumination light L towards the mirror array device 13.

The light guide element 12 is an elongated optical member, such as an image guide fiber, for guiding the illumination light L in the longitudinal direction. The light guide element 12 guides the illumination light L from the light-incident end 12a at the basal end side to a light-emitting end 12b at the distal end side and emits the illumination light L from the light-emitting end 12b. The end surface of the light-incident end 12a is orthogonal to an optical axis A of the light guide element 12. On the distal end surface of the scope 2, an illumination lens 16 is disposed at a position facing the light-emitting end 12b. The illumination lens 16 diffuses the illumination light L emitted from the light-emitting end 12b and emits the illumination light L towards the subject S.

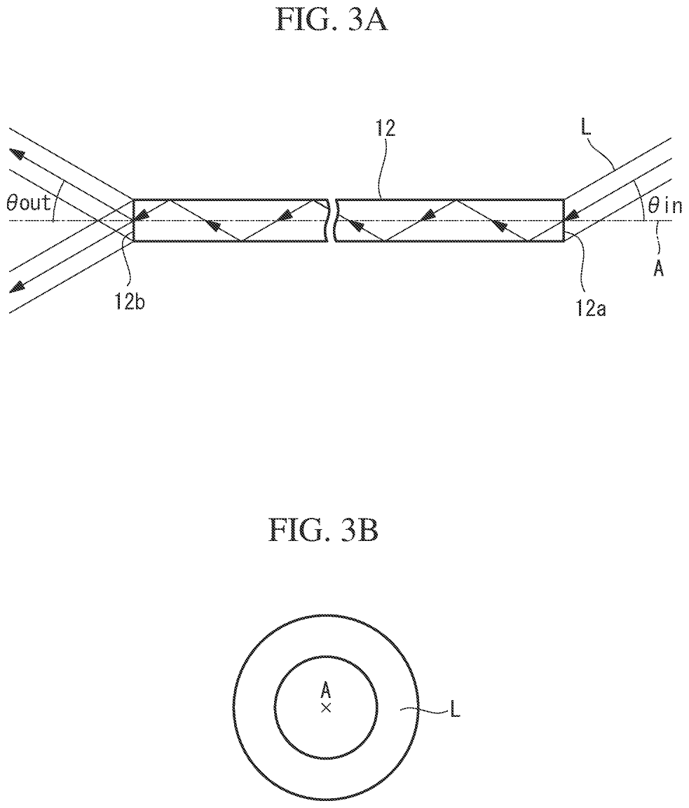

FIGS. 3A and 3B illustrate the relationship between an incident angle θin of light incident on the light-incident end 12a and the light distribution of light emitted from the light-emitting end 12b. As shown in FIG. 3A, the illumination light L travels in the light guide element 12 along the longitudinal direction thereof while being reflected repeatedly. The illumination light L is also guided in the circumferential direction in the light guide element 12. Therefore, the illumination light L emitted from the light-emitting end 12b has an annular belt shape, as shown in FIG. 3B, and the light distribution of the illumination light L emitted from the light-emitting end 12b is symmetric with respect to the center (light distribution angle of 0°). Furthermore, because the angle of the illumination light L relative to the optical axis A of the light guide element 12 is preserved, an emission angle θout of the illumination light L emitted from the light-emitting end 12b becomes equivalent to the incident angle θin of the illumination light L incident on the light-incident end 12a, as shown in FIG. 3A. Therefore, the light distribution of the illumination light L emitted from the light-emitting end 12b varies depending on the incident angle θin.

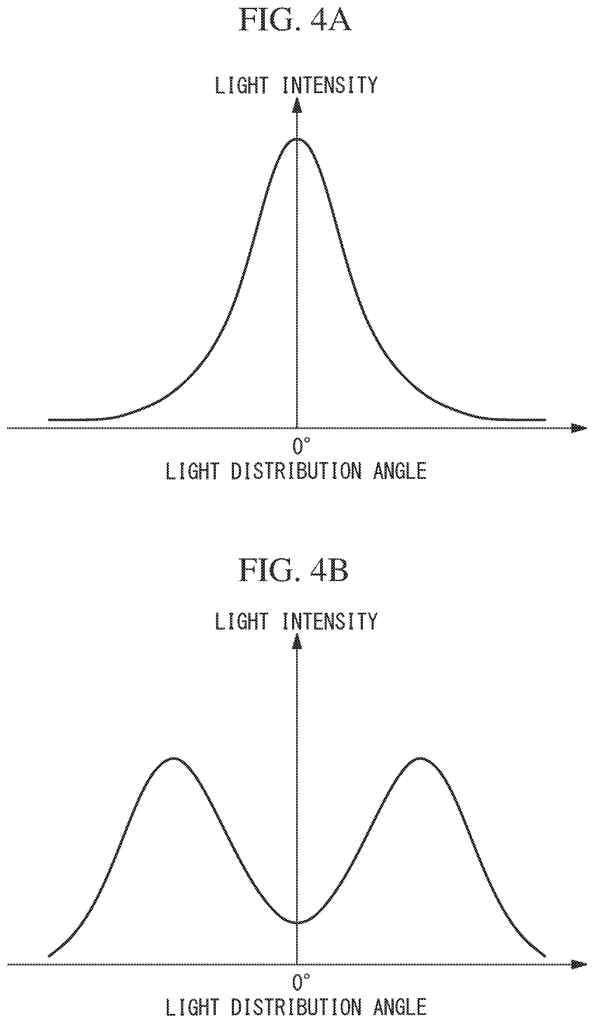

FIGS. 4A and 4B show examples of light distribution characteristics of the illumination light L emitted from the light-emitting end 12b.

The illumination light L emitted from the light source 11 has a light distribution in which the brightness decreases from the center towards the periphery. In the case where the illumination light L is incident on the light-incident end 12a so as to be parallel to the optical axis A (i.e., at an incident angle θin=0°), the light distribution of the illumination light L emitted from the light-emitting end 12b is a narrow light distribution having a peak intensity at the center and exhibiting high directivity, as shown in FIG. 4A, similarly to the light distribution of the illumination light L emitted from the light source 11. In the case where the illumination light L is incident on the light-incident end 12a obliquely relative to the optical axis A, the illumination light L emitted from the light-emitting end 12b forms a wide light distribution having a peak intensity at a peripheral portion, as shown in FIG. 4B. As the incident angle θin becomes larger, the light distribution angle corresponding to the peak intensity moves in a direction further away from 0°, whereby the light distribution of the illumination light L becomes wider (i.e., the diameter of the annular belt-shaped illumination light L in FIG. 3B becomes larger).

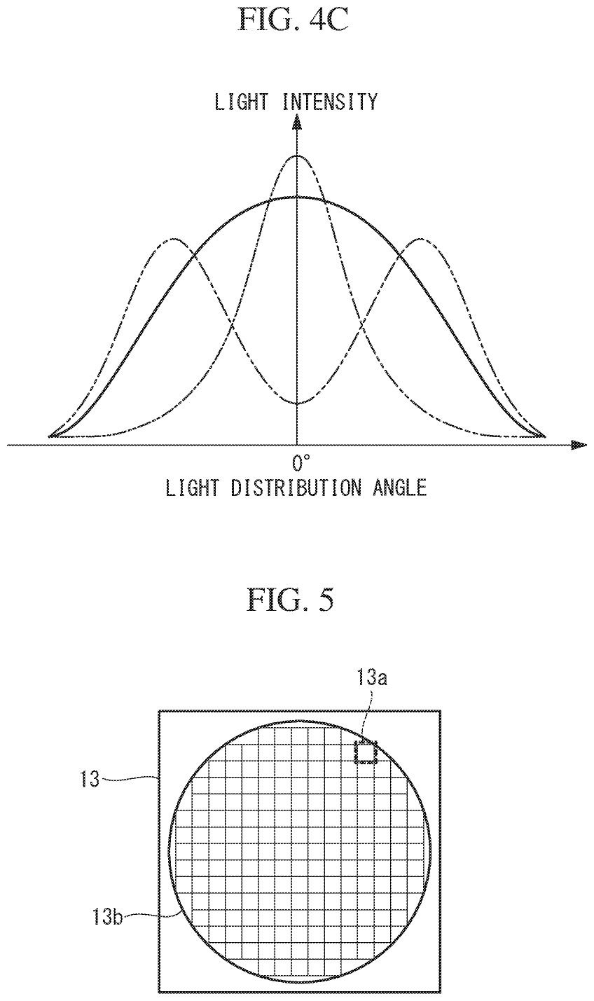

As shown in FIG. 5, the mirror array device 13 is a digital micromirror device (DMD®) having a two-dimensional array composed of a plurality of micromirrors 13a. In FIG. 5, one small rectangle represents one micromirror 13a. The mirror array device 13 has a deflecting surface 13b that receives the illumination light L from the light source 11 and that deflects the illumination light L. The micromirrors 13a are arrayed in two directions orthogonal to each other on the deflecting surface 13b.

The mirror array device 13 changes the angle of each of the micromirrors 13a to either an on angle (first angle) or an off angle (second angle), thereby changing the deflection angle of the illumination light L at each position on the deflecting surface 13b to either a first deflection angle θ1 or a second deflection angle θ2. Therefore, the mirror array device 13 deflects, at the first deflection angle θ1, a portion (first portion) of the illumination light L that has been incident thereon and, at the same time, deflects, at the second deflection angle θ2, another portion (second portion) of the illumination light L that has been incident thereon. Hereinafter, a portion of the illumination light L deflected at the first deflection angle θ1 by micromirrors 13a with the on angle is referred to as on-light Lon, and another portion of the illumination light L deflected at the second deflection angle θ2 by micromirrors 13a with the off angle is referred to as off-light Loff.

The incident angle θin of the illumination light L, which comes from the mirror array device 13 and which is incident on the light-incident end 12a via the lens group 14, is determined according to the deflection angles θ1 and θ2 of the illumination light L deflected by the mirror array device 13. The on-light Lon passes through the lens group 14 and is incident on the light-incident end 12a substantially parallel to the optical axis A (substantially orthogonal to the end surface of the light-incident end 12a). The off-light Loff passes through the lens group 14 and is incident on the light-incident end 12a obliquely relative to the optical axis A. Therefore, as shown in FIG. 4C, the light distribution of the illumination light L emitted from the light-emitting end 12b is equivalent to a combination of the light distribution in FIG. 4A and the light distribution in FIG. 4B. FIG. 4A shows a light distribution in the case where the entire illumination light L that has been incident on the mirror array device 13 is deflected as the on-light Lon, and FIG. 4B shows a light distribution in the case where the entire illumination light L that has been incident on the mirror array device 13 is deflected as the off-light Loff. The on-light Lon illuminates mainly the center portion of the field of view of the image-capturing unit 4, whereas the off-light Loff illuminates mainly a peripheral portion of the field of view of the image-capturing unit 4.

The mirror array device 13 can control the angles of the micromirrors 13a individually and can arbitrarily control the ratio between the number of micromirrors 13a with the on angle and the number of micromirrors 13a with the off angle. Therefore, the mirror array device 13 can change the ratio of the amount of light between the on-light Lon and the off-light Loff to any value, thereby changing the light distribution of the illumination light L emitted from the light-emitting end 12b.

The lens group 14 guides both on-light Lon and off-light Loff from the mirror array device 13 to the light-incident end 12a. In the drawings, the lens group 14 includes a pair of lenses. The lens at the mirror array device 13 side receives both on-light Lon and off-light Loff from the mirror array device 13, and the lens at the light guide element 12 side emits on-light Lon and off-light Loff towards the light-incident end 12a.

The control unit 5 receives an image of the subject S from the image-capturing unit 4 and controls the mirror array device 13 on the basis of the image.

For example, the control unit 5 detects halation and a bright spot in the image on the basis of pixel values. Halation refers to a phenomenon in which a portion of the image appears white as a result of the subject S in the vicinity of the distal end of the scope 2 being irradiated with excessively intense illumination light L. A bright spot refers to a small white spot occurring as a result of regular reflection light of the illumination light L from a surface of the subject S being incident on the image-capturing unit 4. For example, the control unit 5 detects, as halation or a bright spot, a region having pixel values equal to or larger than a predetermined threshold value. Next, the control unit 5 changes the ratio between the number of micromirrors 13a with the on angle and the number of micromirrors 13a with the off angle so as to decrease the brightness of the illumination light L in the region corresponding to the detected halation or bright spot.

The control unit 5 is a processor, such as a central processing unit (CPU). The control unit 5 detects the above-described halation and bright spot according to an image processing program stored in a storage device (not shown in the figure) and controls the mirror array device 13 according to a control program stored in the storage device.

Next, the operation of the illuminating device 1 and the endoscope system 100 with the above-described structure will be described.

According to the endoscope system 100 of this embodiment, illumination light L in the form of a diverging beam emitted from the light source 11 is formed into substantially collimated light by the parabolic surface mirror 15 and is then deflected as on-light Lon and off-light Loff by the mirror array device 13. The on-light Lon and off-light Loff passing through the lens group 14 enter the light guide element 12 via the light-incident end 12a. Illumination light L in the form of the on-light Lon and off-light Loff overlapping each other is emitted from the light-emitting end 12b of the light guide element 12, and the subject S is irradiated with the illumination light L via the illumination lens 16.

The illumination light L reflected at the subject S is received by the image-forming lens 4a. An image of the subject S formed by the image-forming lens 4a is captured by the image sensor 4b, and the image of the subject S is transmitted from the image sensor 4b to the control unit 5.

The control unit 5 checks for halation and a bright spot in the image. If halation or a bright spot is detected, the control unit 5 changes the ratio of the amount of light between the on-light Lon and the off-light Loff by changing the ratio between the number of micromirrors 13a with the on angle and the number of micromirrors 13a with the off angle of the mirror array device 13, thereby decreasing the brightness of the illumination light L in the region suffering from the halation or bright spot.

When, for example, the interior of an elongated lumen, such as the bowel, is observed with the scope 2, halation occurs at a peripheral portion of the image as a result of the cavity wall in the vicinity of the distal end of the scope 2 being irradiated with intense illumination light L. The control unit 5 increases the amount of the on-light Lon by increasing the number of micromirrors 13a with the on angle and reduces the amount of the off-light Loff by reducing the number of micromirrors 13a with the off angle. By doing so, the halation is suppressed as a result of the brightness of the peripheral portion in the image being decreased, and the brightness at a center portion in the image is also increased.

When regular reflection light occurring on a surface of the subject S is incident on the image-forming lens 4a, a bright spot occurs in the image. In the case where a bright spot occurs at the center portion in the image, the control unit 5 reduces the number of micromirrors 13a with the on angle and increases the number of micromirrors 13a with the off angle. In the case where a bright spot occurs at a peripheral portion in the image, the control unit 5 increases the number of micromirrors 13a with the on angle and reduces the number of micromirrors 13a with the off angle. By doing so, the brightness of the illumination light L at the position at which regular reflection light occurs is reduced, whereby the bright spot is suppressed.

In this manner, according to this embodiment, the illumination light L from the light source 11 is deflected by the mirror array device 13 simultaneously at two deflection angles that differ from each other, and two light beams Lon and Loff are incident on the light-incident end 12a at incident angles that differ from one another. As a result of the ratio of the amount of light between these two light beams Lon and Loff being changed by the mirror array device 13, the light distribution of the illumination light L for irradiating the field of view of the image-capturing unit 4 is dynamically adjusted while the subject S is being observed. This provides an advantage in that the subject S can be appropriately illuminated according to the image capturing conditions and the type of the subject, whereby images with excellent image quality can be produced by suppressing halation and bright spots.

In addition, the entire illumination light L that has come from the light source 11 and has been incident on the mirror array device 13 is deflected by the mirror array device 13 simultaneously at two deflection angles, and all of the deflected light Lon and all of the deflected light Loff are simultaneously incident on the light-incident end 12a via the lens group 14, thus irradiating the subject S. In this manner, there is an advantage in that the illumination light L emitted from the light source 11 can be used to irradiate the subject S without loss.

The larger the incident angle θin of the off-light Loff that is incident on the light-incident end 12a, the wider the light distribution of the illumination light L emitted from the light-emitting end 12b. In order to brightly illuminate a peripheral portion of the field of view of the wide-angle image-forming lens 4a with the off-light Loff, the incident angle θin of the off-light Loff is preferably 15° or more.

The larger the number of micromirrors 13a in the two-dimensional array, the higher the resolving power with which the light distribution of the illumination light L emitted from the light-emitting end 12b can be adjusted. Therefore, the two-dimensional array of the micromirrors 13a should preferably have at least 6×6 micromirrors 13a.

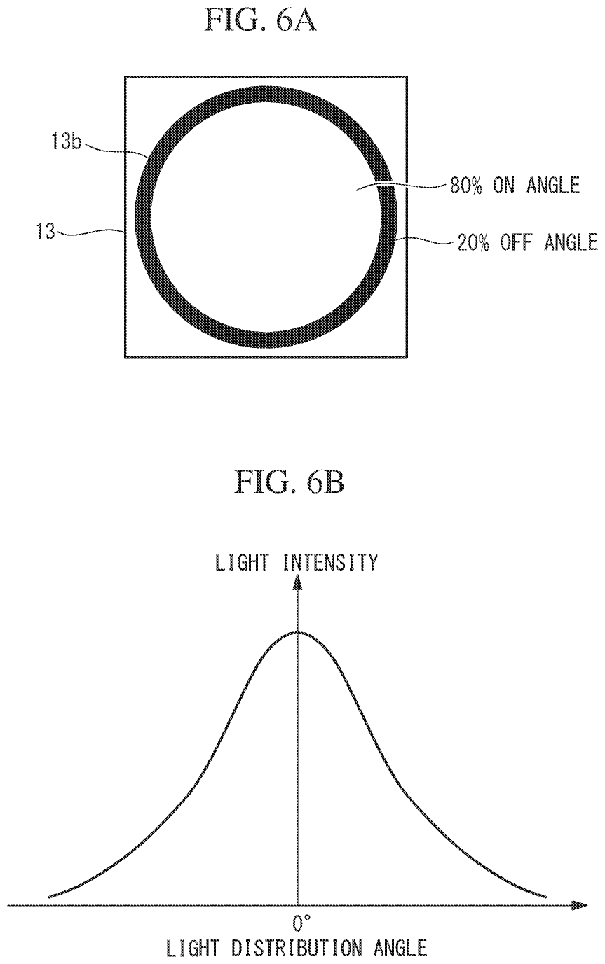

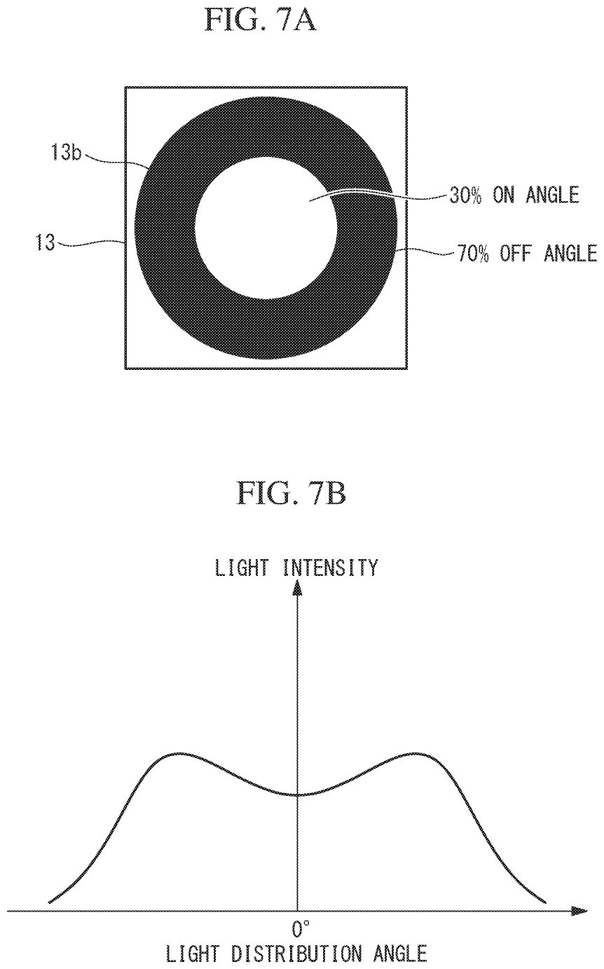

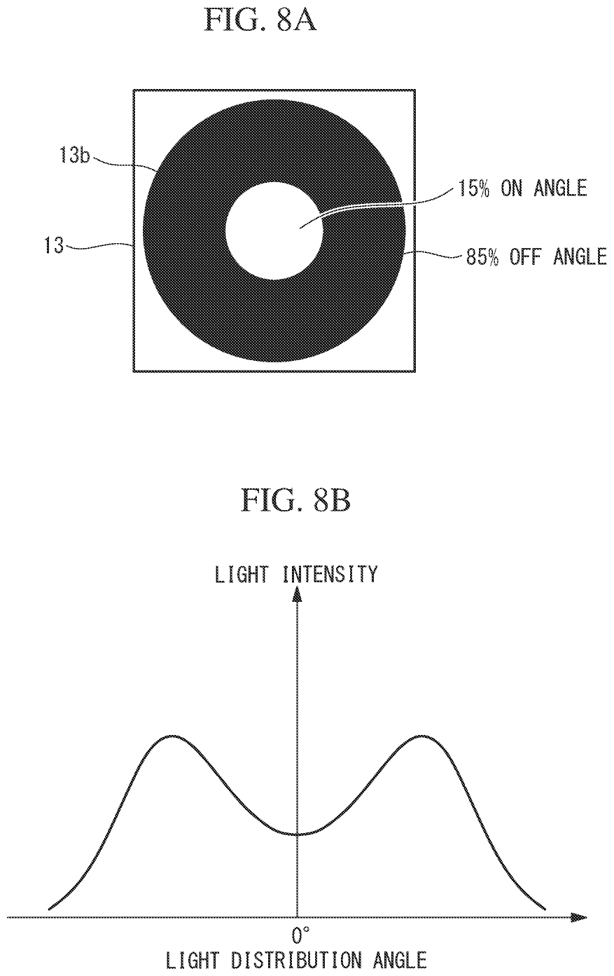

FIGS. 6A, 7A, and 8A show examples in which the mirror array device 13 is controlled by the control unit 5. As shown in FIGS. 6A, 7A, and 8A, the control unit 5 may divide the two-dimensional array into two regions, thereby controlling all the micromirrors 13a in one of the regions (white region in the deflecting surface 13b) to the on angle and controlling all the micromirrors 13a in the other of the regions (black region in the deflecting surface 13b) to the off angle.

In FIGS. 6A, 7A, and 8A, the ratios between the number of micromirrors 13a with the on angle and the number of micromirrors 13a with the off angle are 80:20, 30:70, and 15:85, respectively. FIGS. 6B, 7B, and 8B show light distributions of illumination light L generated with the settings shown in FIGS. 6A, 7A, and 8A.

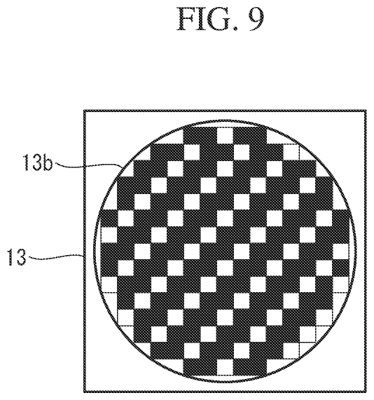

FIG. 9 shows another example in which the mirror array device 13 is controlled by the control unit 5. In FIG. 9, the white regions represent the micromirrors 13a with the on angle, and the black regions represent the micromirrors 13a with the off angle. The control unit 5 may control the micromirrors 13a at arbitrary positions on the two-dimensional array to the on angle and control the micromirrors 13a at other positions to the off angle. In this manner, the light distribution of the illumination light L may be controlled by controlling the ratio between the number of micromirrors 13a with the on angle and the number of micromirrors 13a with the off angle over all the micromirrors 13a of the mirror array device 13.

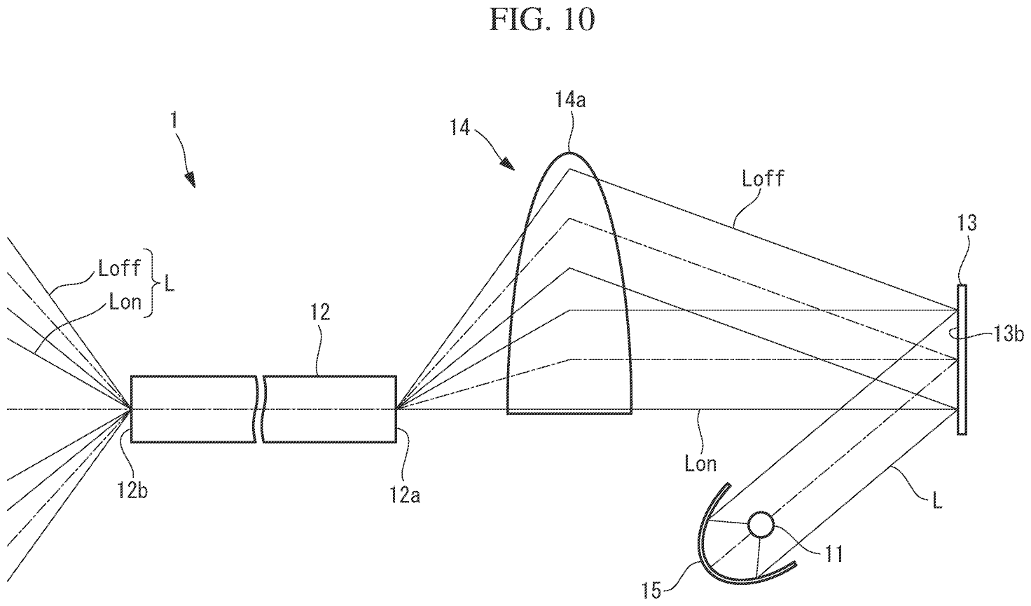

In this embodiment, as shown in FIG. 10, a lens of the lens group 14 may be a semispherical lens 14a the lens surface of which is semicircular or substantially semicircular.

The focal point of the semispherical lens 14a is disposed at the light-incident end 12a, and both on-light Lon and off-light Loff from the mirror array device 13 are focused onto the light-incident end 12a by the semispherical lens 14a.

In this manner, the configuration of the lens group 14 can be simplified by using the semispherical lens 14a. In addition, each of the on-light Lon and the off-light Loff that are incident on the light-incident end 12a is not collimated light but converging light and, therefore, includes beams with various incident angles. For this reason, the light distribution of the illumination light L emitted from the light-emitting end 12b becomes wider than in the case where substantially collimated light is incident on the light-incident end 12a.

Although a DMD is used as the mirror array device 13 in this embodiment, instead of this, another mirror array device that includes a plurality of mirrors arranged one-dimensionally or two-dimensionally and that is capable of individually controlling the angles of the plurality of mirrors may be used. In addition, the present invention may be configured to deflect the illumination light L from the light source 11 simultaneously at three or more deflection angles by means of the mirror array device and to cause three illumination light beams to be incident on the light-incident end 12a at incident angles that differ from one another.

In addition, instead of the mirror array device, a liquid crystal device for controlling the deflection angle of light according to the index of refraction of the liquid crystal may be used as the deflecting element.

Although the ratio of the amount of light between the on-light Lon and the off-light Loff is automatically controlled by the control unit 5 in this embodiment, instead of this, the user may control the ratio.

For example, the user determines the ratio of the amount of light between the on-light Lon and off-light Loff on the basis of the image of the subject S displayed on the display device and inputs the ratio of the amount of light to the control unit 5 by using an input device (not shown in the figure) connected to the control unit 5. The control unit 5 controls the mirror array device 13 according to the ratio of the amount of light input by the user.

By doing so, while observing an image of the subject S, the user can adjust, to a desired light distribution, the light distribution of the illumination light L for irradiating the subject S.

As a result, the above-described embodiment leads to the following aspects.

One aspect of the present invention is directed to an illuminating device including: a light source; a light guide element that has a light-incident end and a light-emitting end, the light guide element guiding illumination light incident on the light-incident end to emit the illumination light from the light-emitting end; and a deflecting element that has a deflecting surface for deflecting the illumination light coming from the light source towards the light-incident end of the light guide element and that makes the illumination light incident on the light-incident end at an incident angle according to a deflection angle, wherein the deflecting element can change, between a first deflection angle and a second deflection angle, the deflection angle of the illumination light at each position on the deflecting surface and deflects the illumination light simultaneously at the first deflection angle and the second deflection angle, and a first portion of the illumination light, the first portion being deflected at the first deflection angle, and a second portion of the illumination light, the second portion being deflected at the second deflection angle, are both incident on the light-incident end.

According to this aspect, illumination light emitted from the light source is deflected at the deflecting surface of the deflecting element, is incident on the light-incident end of the light guide element, and is emitted from the light-emitting end of the light guide element, thus irradiating a subject. The emission angle of the illumination light emitted from the light-emitting end depends on the incident angle of the illumination light that is incident on the light-incident end, and the incident angle of the illumination light that is incident on the light-incident end is controlled according to the deflection angle of the illumination light deflected by the deflecting element. Therefore, it is possible to control the light distribution of the illumination light that irradiates the subject according to the deflection angle of the illumination light deflected by the deflecting element.

In this case, because the illumination light is deflected by the deflecting element simultaneously at the first deflection angle and the second deflection angle, the light distribution of the illumination light that irradiates the subject is a combination of two light distributions. Therefore, it is possible to achieve various light distributions by changing the deflection angle of the illumination light at each position on the deflecting surface, thereby making it possible to appropriately illuminate the subject according to image capturing conditions and the type of the subject. In addition, because the first portion and the second portion of the illumination light are both incident on the light-incident end of the light guide element when the subject is illuminated, it is possible to prevent loss of the amount of the illumination light.

In the above-described aspect, the deflecting element may be capable of changing a ratio of an amount of light between the first portion and the second portion.

This configuration allows for more various light distributions of the illumination light.

In the above-described aspect, the deflecting element may include a mirror array device having an array composed of a plurality of mirrors and may be capable of individually changing the angles of the plurality of mirrors.

The loss of the illumination light emitted from the light source can be further reduced by using, as the deflecting element, mirrors with reduced loss in the amount of light.

In the above-described aspect, the mirror array device may have a two-dimensional array composed of a plurality of mirrors, and the two-dimensional array may include six or more of the mirrors being arranged in a first direction and six or more of the mirrors being arranged in a second direction orthogonal to the first direction.

This configuration allows the light distribution of the illumination light to be adjusted with even higher resolving power.

In the above-described aspect, the mirror array device may be capable of changing an angle of each of the plurality of mirrors to either a first angle or a second angle and a number of mirrors with the first angle and a number of mirrors with the second angle are changeable.

The amount of light of the first portion and the amount of light of the second portion can be changed by changing the number of mirrors with the first angle and the number of mirrors with the second angle, whereby it is possible to change the light distribution of the illumination light.

In the above-described aspect, the mirror array device may change an angle of each of the plurality of mirrors to either the first angle or the second angle, the first portion of the illumination light, the first portion being deflected by the mirrors with the first angle, may be incident on an end surface of the light-incident end substantially orthogonal thereto, and the second portion of the illumination light, the second portion being deflected by the mirrors with the second angle, may be incident on the end surface of the light-incident end at an incident angle of 15° or more relative thereto.

The brightness at the center portion of illumination light emitted from the light-emitting end of the light guide element can be controlled by means of the amount of light of the first portion deflected by mirrors with the first angle. The brightness at a peripheral portion of illumination light emitted from the light-emitting end of the light guide element can be controlled by means of the second portion deflected by mirrors with the second angle. In addition, as a result of the incident angle of the second portion that is incident on the light-incident end being 15° or more, a peripheral portion of a wide angle of field of view, such as the field of view of an endoscope, can be illuminated brightly.

Another aspect of the present invention is directed to an endoscope system including: one of the above-described illuminating devices; and an image-capturing unit that captures an image of a subject illuminated with illumination light emitted from the light-emitting end of the light guide element.

The above-described aspect may further include: a control unit that controls the deflecting element on a basis of the image of the subject.

This configuration allows the light distribution of the illumination light to be automatically adjusted such that the brightness at each portion of the image becomes appropriate.

The present invention affords an advantage in that a subject can be appropriately illuminated according to image capturing conditions and the type of the subject and loss in the amount of the illumination light can be prevented.

### REFERENCE SIGNS LIST

1 Illuminating device
2 Scope
3 Light source device
4 Image-capturing unit
5 Control unit
6 Illumination lens
11 Light source
12 Light guide element
12a Light-incident end
12b Light-emitting end
13 Mirror array device
13a Micromirror (mirror)
13b Deflecting surface
14 Lens group
15 Parabolic surface mirror
100 Endoscope system
L Illumination light
Lon On-light (first portion of illumination light)
Loff Off-light (second portion of illumination light)
θ1 First deflection angle
θ2 Second deflection angle
θin incident angle

1. An illuminating device comprising:
a light source;
a light guide element that has a light-incident end and a light-emitting end, the light guide element guiding illumination light incident on the light-incident end to emit the illumination light from the light-emitting end; and
a deflecting element that has a deflecting surface for deflecting the illumination light coming from the light source towards the light-incident end of the light guide element and that makes the illumination light incident on the light-incident end at an incident angle according to a deflection angle,
wherein the deflecting element is configured to change, between a first deflection angle and a second deflection angle, the deflection angle of the illumination light at each position on the deflecting surface and deflects the illumination light simultaneously at the first deflection angle and the second deflection angle, and
a first portion of the illumination light, the first portion being deflected at the first deflection angle, and a second portion of the illumination light, the second portion being deflected at the second deflection angle, are both incident on the light-incident end.

2. The illuminating device according to claim 1, wherein the deflecting element is configured to change a ratio of an amount of light between the first portion and the second portion.

3. The illuminating device according to claim 1, wherein the deflecting element comprises a mirror array device having an array composed of a plurality of mirrors and is configured to individually change angles of the plurality of mirrors.

4. The illuminating device according to claim 3,
wherein the mirror array device has a two-dimensional array composed of a plurality of mirrors, and
the two-dimensional array comprises six or more of the mirrors arranged in a first direction and six or more of the mirrors arranged in a second direction orthogonal to the first direction.

5. The illuminating device according to claim 3,
wherein the mirror array device is configured to change an angle of each of the plurality of mirrors to either a first angle or a second angle and
a number of mirrors with the first angle and a number of mirrors with the second angle are changeable.

6. The illuminating device according to claim 3,
wherein the mirror array device changes an angle of each of the plurality of mirrors to either the first angle or the second angle,
the first portion of the illumination light, the first portion being deflected by the mirrors with the first angle, is incident on an end surface of the light-incident end substantially orthogonal thereto, and
the second portion of the illumination light, the second portion being deflected by the mirrors with the second angle, is incident on the end surface of the light-incident end at an incident angle of 15° or more relative thereto.

7. An endoscope system comprising:
the illuminating device according to claim 1; and
an image-capturing unit that captures an image of a subject illuminated with illumination light emitted from the light-emitting end of the light guide element.

8. The endoscope system according to claim 7, further comprising: a control unit that controls the deflecting element on a basis of the image of the subject.
